(19) United States
(12) Patent Application Publication
(10) Pub. No.: US 2023/0076898 A1
(43) Pub. Date: Mar. 9, 2023

(54) SHOE AND INSOLE MAPPING APPARATUS

(71) Applicant: AHARONI PELEG HAIM, MOSHAV MAZOR (IL)

(72) Inventor: AHARONI PELEG HAIM, MOSHAV MAZOR (IL)

(21) Appl. No.: 17/983,422

(22) Filed: Nov. 9, 2022

Related U.S. Application Data
(63) Continuation-in-part of application No. PCT/IL2021/050540, filed on May 12, 2021.
(60) Provisional application No. 63/024,522, filed on May 14, 2020.

(51) Int. Cl. A61B 5/103; A61B 5/00

(52) U.S. Cl. CPC A61B 5/1038; A61B 5/6807; A61B 5/486

(57) ABSTRACT

An insole mapping apparatus (10C), including: at least one location sensor (20C) and potentially two or more location sensors such as (20A,20B) each for being attached to one organ of the user (19), for measuring locations (21A,21B,21C) of the organs in relation to at least one stationary object (26A); and a processor (29), for determining deformations (36A1) of a body of the user (19) according to the measurements (21A,21B,21C), and for determining an insole map (74) being opposite to the determined deformations (36A1).

### TECHNICAL FIELD

The invention relates to the field of shoes and insoles.

### BACKGROUND

Nowadays, insole mapping is produced by measuring the user's foot surface and by other techniques. Then, the map is converted to produce an insole having various heights according to the map.

However, this mapping does not provide good balancing, and further, the produced insole cannot be further adjusted.

There is a long felt need to provide a solution to the above-mentioned and other problems of the prior art.

### SUMMARY

An insole mapping apparatus, including:

location sensors; and

a processor (29),

for determining deformations (36A1) and an insole map.

### BRIEF DESCRIPTION OF THE DRAWINGS

Embodiments, features, and aspects of the invention are described herein in conjunction with the following drawings:

FIG. 1 depicts an insole mapping apparatus according to one embodiment of the invention.

FIG. 2 depicts an example of the shoes having equal insoles, on which the user of FIG. 1 is standing.

FIG. 3 describes a first measurement applied by the sensors of FIG. 1 having a non-proper user's body.

FIG. 4 describes the deformation concluded from the measurements of FIG. 3.

FIG. 5 describes the insole map, for correcting the roll deformation of FIG. 4.

FIG. 6 describes the measurement of FIG. 3 upon correcting the insoles to the insole map of FIG. 5.

FIG. 7 describes the corrected state of the user, concluded from the measurements of FIG. 6.

FIG. 8 describes distance measurements determining pitch deformation.

FIG. 9 describes the insole map, for correcting the pitch deformation of FIG. 8.

FIG. 10 describes distance measurements determining pitch deformation opposite to that of FIG. 8.

FIG. 11 describes the insole map, for correcting the pitch deformation of FIG. 10.

FIG. 12 describes the insole map of the rear of the right shoe, for correcting the yaw deformation indicated by the arrow of FIG. 1.

FIG. 13 is a perspective view of the right shoe of FIG. 1.

FIG. 14 is a sectional side view of the right shoe of FIG. 13 and the stationary pattern of FIG. 1 disposed apart.

The drawings are not necessarily drawn to scale.

### DETAILED DESCRIPTION

The invention will be understood from the following detailed description of embodiments of the invention, which are meant to be descriptive and not limiting. For the sake of brevity, some well-known features are not described in detail.

The reference numbers have been used to point out elements in the embodiments described and illustrated herein, in order to facilitate the understanding of the invention. They are meant to be merely illustrative, and not limiting. Also, the foregoing embodiments of the invention have been described and illustrated in conjunction with systems and methods thereof, which are meant to be merely illustrative, and not limiting.

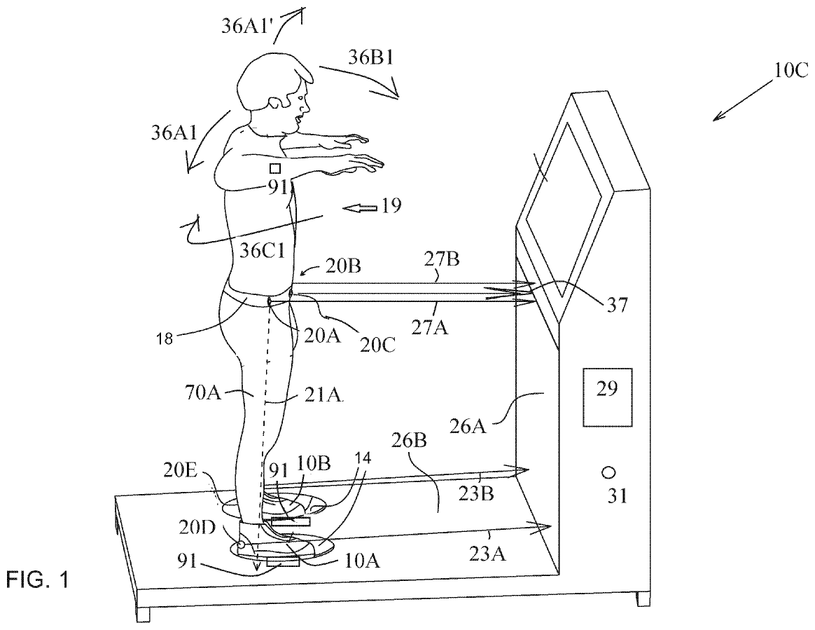

FIG. 1 depicts an insole mapping apparatus according to one embodiment of the invention.

An insole mapping apparatus 10C according to one embodiment is intended for mapping insoles of shoes 10A and 10B.

Insole mapping apparatus 10C includes two stationary patterns 14, for inserting thereinto the user's feet, or shoes 10A and 10B, while being worn by the user.

Insole mapping apparatus 10C further includes a belt 18 or accessories for being worn by the user. Belt 18 includes location sensors 20A, 20B, 20C, 20D and 20E, such as distance sensors for measuring distances of the user's organs as worn by belt, to stationary objects 26A and 26B of insole mapping apparatus 10C.

Location sensors 20A, 20B, 20C, 20D and 20E may further include markers, such as lasers, and may further measure angles as indicated by the markers

Insole mapping apparatus 10C further includes a processor 29 for processing the measured distances and angles to a map of insoles.

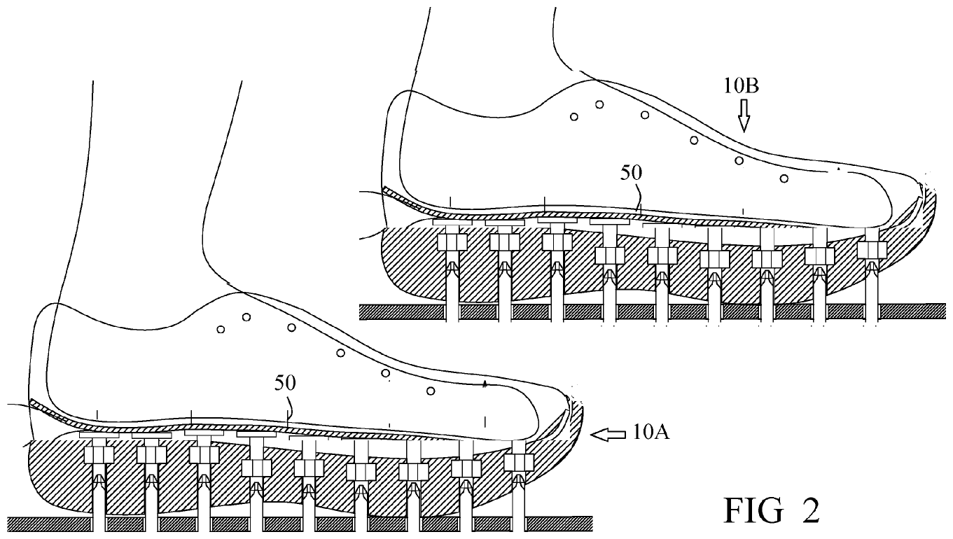

FIG. 2 depicts an example of shoes 10A and 10B having equal insoles, on which the user of FIG. 1 is standing.

Insoles 50 of both shoes of FIG. 2 are equal.

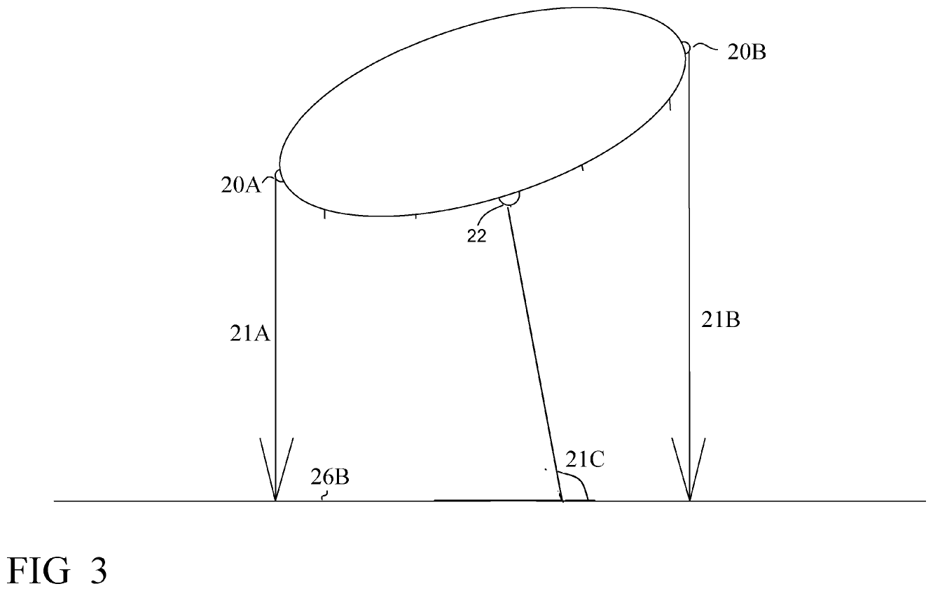

FIG. 3 describes a first measurement applied by the sensors of FIG. 1 having a non-proper user's body.

Right chest sensor 20A measures a vertical distance 21A therefrom to the floor 26B; and left chest sensor 20B measures a vertical distance 21B therefrom to the floor 26B.

In the example of the user wearing shoes 10A and 10B of FIG. 2 having equal insoles 50, right chest sensor 20A has measured vertical distance 21A being smaller than vertical distance 21B measured by left chest sensor 20B.

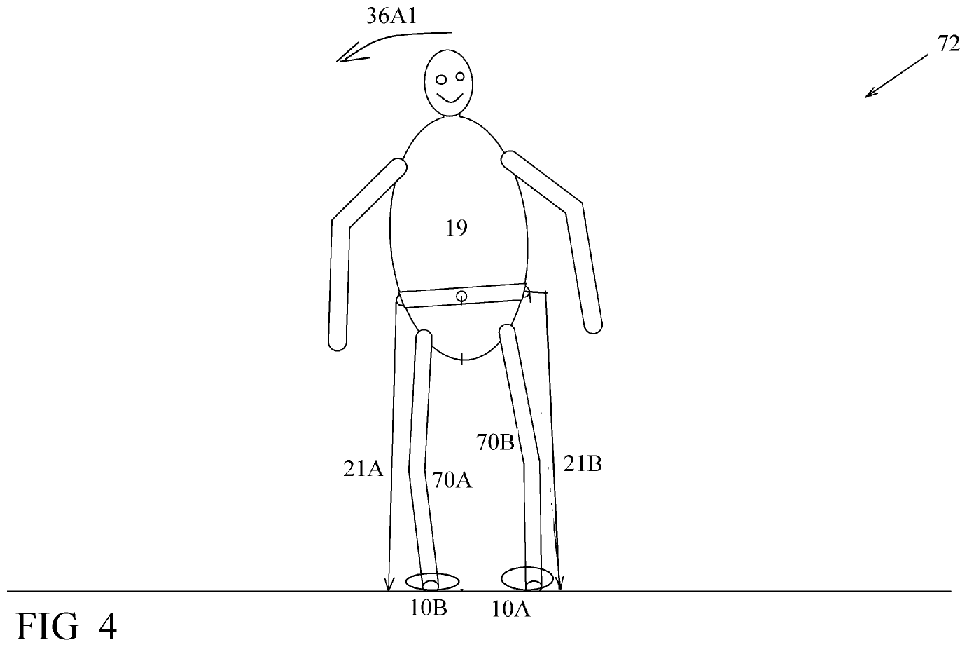

FIG. 4 describes the deformation concluded from the measurements of FIG. 3.

Processor 29 of FIG. 1 concludes from vertical distance 21A being smaller than vertical distance 21B of FIG. 3, or from angle 21C measured by center sensor 20C not being right angled, a non-balanced standing, named here deformation, that the user's right leg 70A is shorter than the left leg 70B.

The deformation of body of user 19 for this case of measurement is enumerated 36A1 and named herein “roll deformation”.

Controller 29 is programmed that vertical distance 21A smaller than vertical distance 21B provides roll deformation 36A1.

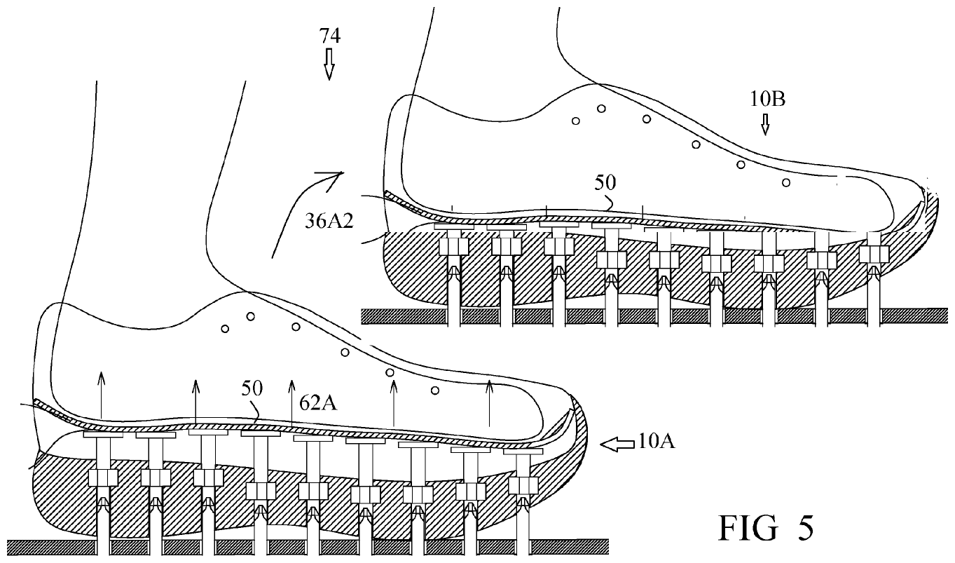

FIG. 5 describes the insole map, for correcting the roll deformation of FIG. 4.

Standing of user 19 diagnosed to roll deformation 36A1 of FIG. 4 is corrected by roll correction 36A2 obtained by an insole map 74 characterized by insole 50 of right shoe 10A being disposed higher (62A) than insole 50 of left shoe 10B, determined by processor 29.

Insole map 74 of FIG. 5 corrects the user's standing, since insole map 74 of roll correction 36A2 directed to insole 50 of right shoe 10A disposed higher (62A) than insole 50 of left shoe 10B, is opposite to user's right leg 70A being shorter than left leg 70B being roll deformation 36A1.

Insole mapping apparatus 10C may further physically produce a traditional insole according to the determined insole map 74, and/to correct shoe 10A to physically lift points of insole 50 in relation to the outsole of shoe 10A.

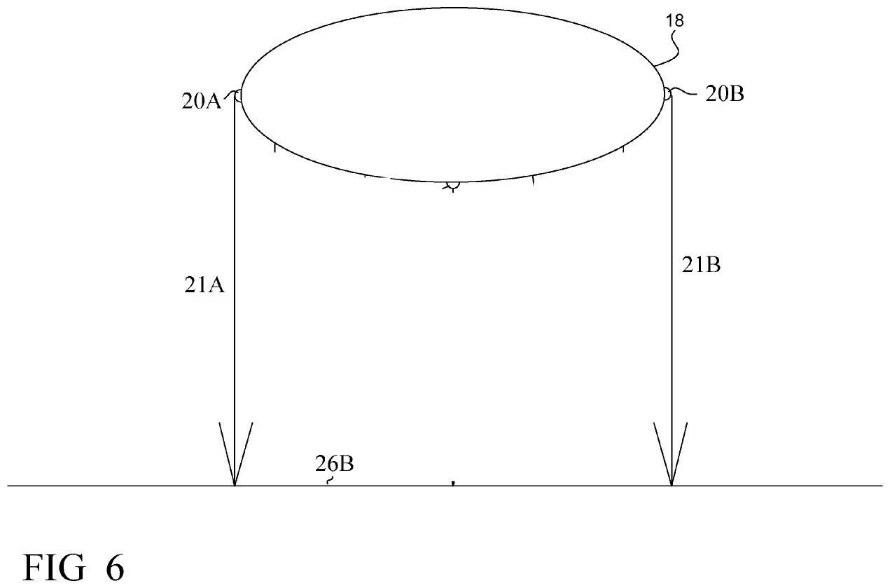

FIG. 6 describes the measurement of FIG. 3 upon correcting the insoles to the insole map of FIG. 5.

Upon applying the correction to the shoes, right chest sensor 20A and left chest sensors 20B measure equal vertical distances 21A and 21B, since roll correction 36A2 canceling roll deformation 36A1.

The physical correction of shoe 10A and the distance measurements may be applied concurrently.

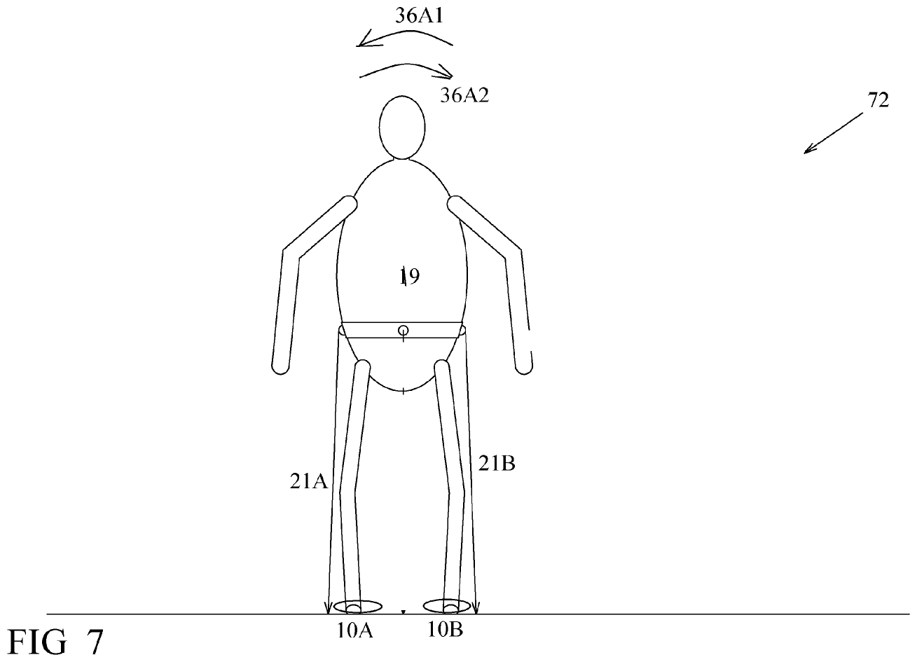

FIG. 7 describes the corrected state of the user, concluded from the measurements of FIG. 6.

Processor 29 of FIG. 1 concludes correct standing 72 from the equal measured vertical distances of FIG. 6.

Location sensors 20A and others may constitute dynamic location sensors, for allowing processor 29 to process locations of the user organs during standing and walking along time.

Insole mapping apparatus 10C may constitute video cameras 31 to process locations of the user organs during standing and walking along time.

Referring again to FIG. 1, FIG. 2 to FIG. 7 describe diagnosing side horizontal deformations 36A1 by insole mapping apparatus 10C, and correcting them. Insole mapping apparatus 10C may as well handle the other 2 axes of deformations.

Side horizontal deformation 36A1 is above named “roll deformation” similar to aircraft principle axes. Following the other aircraft principle axes, deformation 36B1 is herein named “pitch deformation”, and deformation 36C1 is herein named “yaw deformation”.

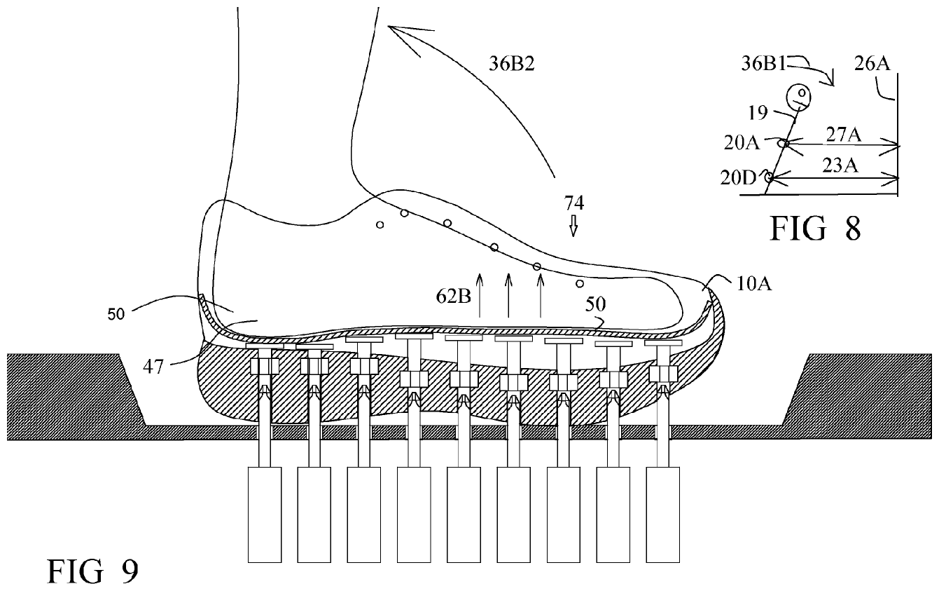

FIG. 8 describes distance measurements determining pitch deformation.

Pitch deformation 36B1 of the user's body may be diagnosed by right chest sensor 20A measuring a horizontal distance 27A being smaller than distance 23A measured by right leg sensor 20D.

FIG. 9 describes the insole map, for correcting the pitch deformation of FIG. 8.

Diagnosed pitch deformation 36B1 of FIG. 8 is corrected by pitch correction 36B2 obtained by an insole map 74 characterized by the front of insole 50 of both shoes 10A and 10B being disposed higher (62B) than normal in relation to the rear of insole 50.

Insole map 74 of FIG. 9 corrects the user's standing, since insole map 74 of pitch correction 36B2 directed to front of insoles 50 being disposed higher (62B) than normal in relation to the rear of insoles 50, is opposite to pitch deformation 36B1.

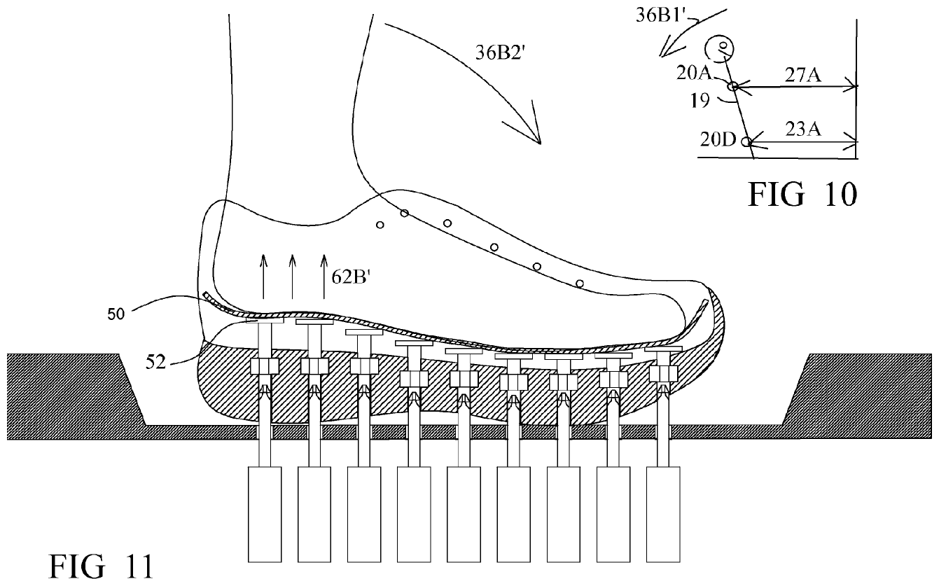

FIG. 10 describes distance measurements determining pitch deformation opposite to that of FIG. 8.

Pitch deformation 36B1′ opposite to pitch deformation 36B1 of FIG. 8 may be diagnosed by horizontal distance 27A being larger than horizontal distance 23A measured by right leg sensor 20D.

FIG. 11 describes the insole map, for correcting the pitch deformation of FIG. 10.

Diagnosed pitch deformation 36B 1′ of FIG. 10 is corrected by pitch correction 36B2′ obtained by an insole map 74 characterized by the rear of insole 50 of both shoes 10A and 10B being disposed higher (62B′) than normal in relation to the front of insole 50.

Referring again to FIG. 1, yaw deformation 36C1 may be diagnosed by right chest sensor 20A measuring a horizontal distance 27A being different from horizontal distance 27B measured by left chest sensor 20B. Yaw deformation 36C1 may alternatively be determined by a center sensor 20C being directed aside the center horizontal point 37 of vertical stationary object 26A.

Yaw deformation 36C1 being clockwise by the arrow of FIG. 1 is caused from lift of the left rear corner of the left foot of user 19.

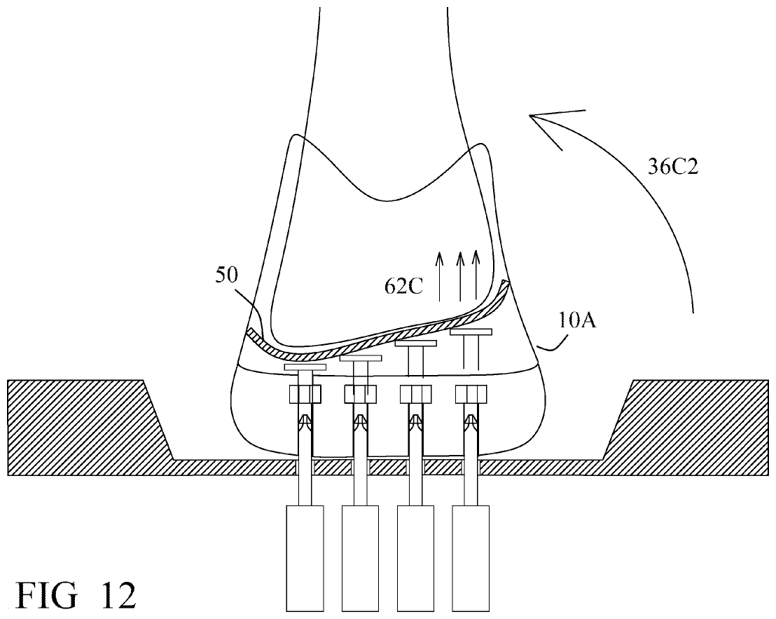

FIG. 12 describes the insole map of the rear of the right shoe, for correcting the yaw deformation indicated by the arrow of FIG. 1.

Diagnosed yaw deformation 36C1 indicated by the arrow of FIG. 12, caused from lifting the left rear corner of the left foot, is corrected by yaw correction 36C2 obtained by an insole map 74 characterized inter alia by the right rear corner of insole 50 of right shoe 10A being disposed higher (62C) than the left rear corner of that support 50.

Insole map 74 of FIG. 12 corrects the user's standing, since insole map 74 of yaw correction 36C2 directed to right rear corner of insole 50 of right shoe 10A being disposed higher (62C) than the left rear corner of that support 50, is opposite to lift of the left rear corner of the left foot being yaw deformation 36C1.

In general, insole map 74 produced by insole mapping apparatus corrects the user's standing, since insole map 74 includes a combination of roll correction 36A1, of pitch correction 36B1, and of yaw correction 36C2 being opposite to the combination of roll deformation 36A1, and of pitch deformation 36A2, and of yaw deformation 36C1.

Insole mapping apparatus 10C may further use biofeedback sensors. Cameras 31 may further diagnose walking patterns. Insole mapping apparatus 10C may further allow the user to manually adjust the lifted points of the insole according to the user's sensation.

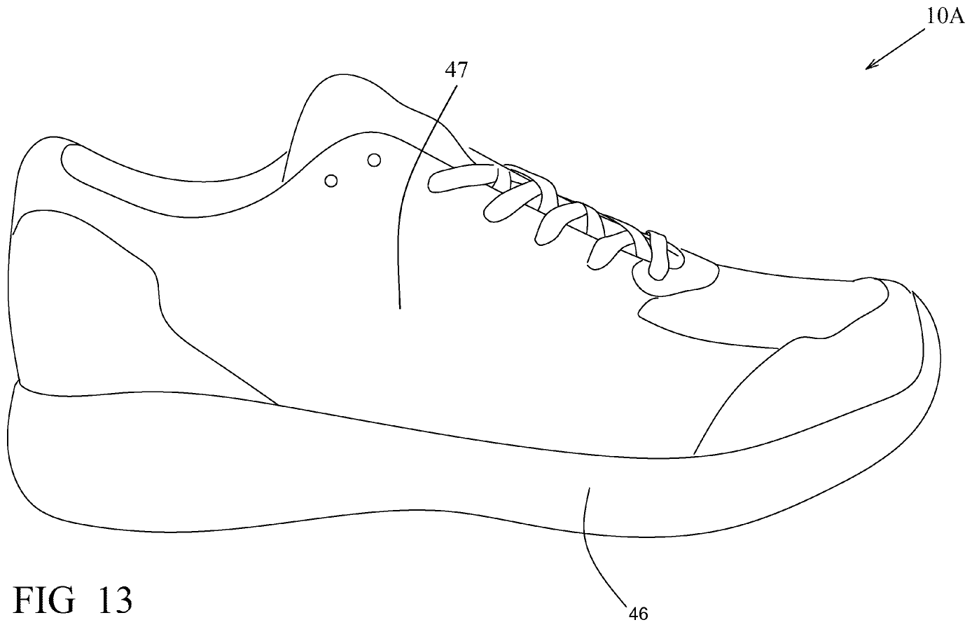

FIG. 13 is a perspective view of the right shoe of FIG. 1.

Shoes 10A and 10B produced by insole mapping apparatus 10C may be normally usable outside insole mapping apparatus 10C.

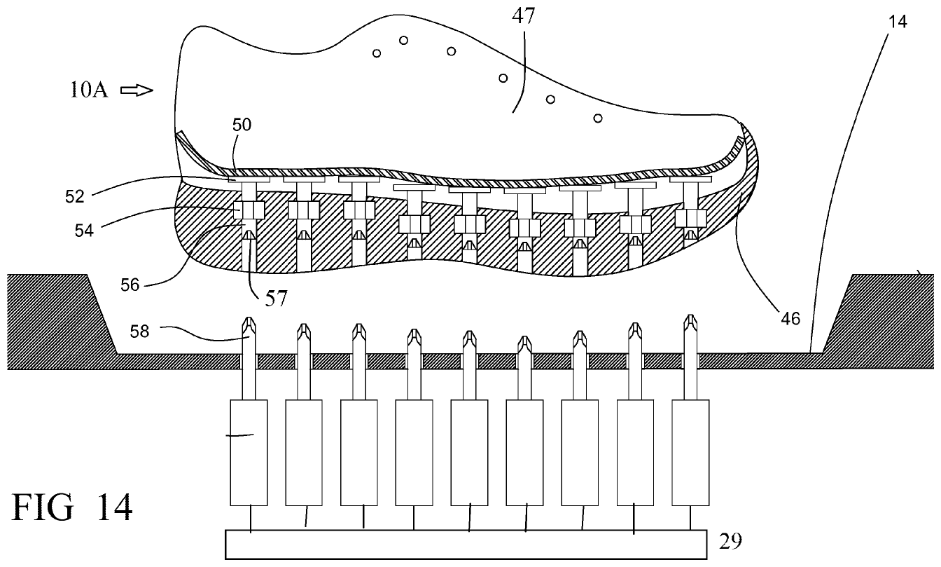

FIG. 14 is a sectional side view of the right shoe of FIG. 13 and the stationary pattern of FIG. 1 disposed apart.

Each of shoes 10A and 10B includes an outsole 46; a plurality of nuts 54 fixed to outsole 46 distributed therearound; a screw 56 threaded through each nut 54; each screw includes a screwing head 57 disposed below its nut 54 and a plate 52 disposed above its nut 54; and insole 50 being liftable by plates 52.

Stationary pattern 14 of insole mapping apparatus 10C may include a plurality of screwdrivers 58, each for being disposed against one of screwing heads 57 of shoe 10A, for rotating screw 56, for lifting and lowering plate 52.

Screwdrivers 58 are controlled by processor 29 of FIG. 1.

Thus, in one aspect, the invention is directed to an insole mapping apparatus (10C), including:
at least one location sensor (20C) and potentially two or more location sensors such as (20A,20B), each for being attached to one organ of the user (19), for measuring locations (21A,21B,21C) of the organs in relation to at least one stationary object (26A); and
a processor (29),
for determining deformations (36A1) of a body of the user (19) according to the measurements (21A,21B), and
for determining an insole map (74) being opposite to the determined deformations (36A1).

The determined deformations include: roll deformation (36A1), pitch deformation (36B1), yaw deformation (36C1) and a combination thereof.

The insole mapping apparatus (10C) may further include:
two stationary patterns (14) fixed to the at least one stationary object (26A), each of the two stationary patterns (14) for inserting a foot of the user (19) thereinto,
thereby the location measuring of the organs and the determined deformations (36A1) relate to known positions of the feet.

The location measuring may be along time.

The insole mapping apparatus (10C) may further include:
a plurality of screwdrivers (58), for being operated by the processor (29) according to the insole map (74).

The insole mapping apparatus (10C) may further include a shoe (10A) including:
an outsole (46);
a plurality of nuts (54) fixed to the outsole (46);
a screw (56) threaded through each of the nuts (54) and including a head (57), being accessible from a bottom of the outsole (46); and
an insole (50) being supported by the screws (56),
thereby allowing disposing the bottom of the outsole (46) on a plurality of the screwdrivers (58) for rotating the screws (56), for lifting the insole (50) supported thereby, in relation to the outsole (46).

The processor (29) may further be configured to determine the deformation (36A1) of the user's body according to a biofeedback sensors (91), or according to manual inputs according sensation of the user (19), or according to cameras (31) diagnosing walking patterns.

Biofeedback sensors 91 of FIG. 1 may be located under the feet of the user for mapping the pressures effects thereon.

In another embodiment, the invention is directed to a shoe (10A), including:
an outsole (46);
a plurality of nuts (54) fixed to the outsole (46);
a screw (56) threaded through each of the nuts (54) and including a head (57), being accessible from a bottom of the outsole (46); and
an insole (50) being supported by the screws (56),
thereby allowing disposing the bottom of the outsole (46) on a plurality of screwdrivers (58) for rotating the screws (56), for lifting the insole (50) supported thereby, in relation to the outsole (46).

The shoe (10A) may further include:
an insole mapping apparatus (10C) for determining an insole map (74), and for operating the screwdrivers (58) according to the insole map (74).

In the figures and/or description herein, the following reference numerals (Reference Signs List) have been mentioned:
numeral 10A and 10B denotes the right and left shoes respectively, according to one embodiment of the invention;
10C: insole mapping apparatus according to one embodiment of the invention;
14: stationary patterns for inserting user's feet which may wear shoes;
18: belt;
19: user;
20A: location sensor disposed on the right chest of user 19;
20B: location sensor disposed on the left chest of user 19;
20C: location sensor disposed on the user's navel;
20D: location sensor disposed on the user's right foot;
20E: location sensor disposed on the user's left foot; these sensors are exemplary;
21A,21B,23A,23B,27A,27B: measured distances;
21C: measure angle;
26A: front wall;
29: processor/controller;
36A1: roll deformation of the user's body, i.e., the user standing tilted aside;
31: camera;
36A1′: pitch deformation opposite to pitch deformation 36A1;
36B1: pitch deformation of the user's body, i.e., the user standing tilted forward or backward;
36B1′: pitch deformation opposite to pitch deformation 36B1;
36C1: pitch deformation of the user's body, i.e., the user standing rotated clockwise or counterclockwise;
36A2: roll correction, i.e., correction to be obtained by insole 50 formed according to insole map 74, for canceling roll deformation 36A1 of the user's body;
36B2: pitch correction, i.e., correction to be obtained by insole 50 formed according to insole map 74, for canceling pitch deformation 36B1 of the user's body;
36B2′: pitch correction opposite to pitch correction 36B2;
36C2: yaw correction, i.e., correction to be obtained by insole 50 formed according to insole map 74, for canceling yaw deformation 36C1 of the user's body;
37: point;
46: outsole;
62A,62B,62B′,62C: lifting of selected pins, screws, or points of insole 50;
70A: right leg;
70B: left leg;
72: standing position;
74: insole map, i.e., height level of the points of the insole, or of the pins or screws;
50: insole;
52: plate for lifting a point of insole 50;
54: nut fixed to outsole;
57: net head, which may be female;
58: screwdriver operated by processor/controller 29.

The foregoing description and illustrations of the embodiments of the invention have been presented for the purpose of illustration, and are not intended to be exhaustive or to limit the invention to the above description in any form.

Any term that has been defined above and used in the claims, should be interpreted according to this definition.

The reference numbers in the claims are not a part of the claims, but rather used for facilitating the reading thereof. These reference numbers should not be interpreted as limiting the claims in any form.

What is claimed is:

1. An insole mapping apparatus (10C), comprising:
at least one location sensor (20C) for being attached to one organ of a user (19), for measuring locations (21A,21B,21C) of said organs in relation to at least one stationary object (26A); and
a processor (29),
for determining at least one deformation (36A1) of a body of said user (19) according to said measurements (21A,21B,21C), and
for determining an insole map (74) being opposite to said at least one determined deformation (36A1).

2. The insole mapping apparatus (10C) of claim 1, wherein said determined deformations comprise at least one member selected from a group consisting of: roll deformation (36A1), pitch deformation (36B1), yaw deformation (36C1) and a combination thereof.

3. The insole mapping apparatus (10C) according to claim 1, further comprising:
two stationary patterns (14) fixed to said at least one stationary object (26A), each of said two stationary patterns (14) for inserting a foot of said user (19) thereinto,
thereby said location measuring of said organs and said determined deformations (36A1) relate to known positions of said feet.

4. The insole mapping apparatus (10C) according to claim 1, wherein said location measuring is along walking time.

5. The insole mapping apparatus (10C) according to claim 1, further comprising:
a plurality of screwdrivers (58), for being operated by said processor (29) according to said insole map (74).

6. The insole mapping apparatus (10C) according to claim 1, further comprising a shoe (10A), comprising:
an outsole (46);
a plurality of nuts (54) fixed to said outsole (46);
a screw (56) threaded through each of said nuts (54) and comprising a head (57), being accessible from a bottom of said outsole (46); and
an insole (50) being supported by said screws (56),
thereby allowing disposing said bottom of said outsole (46) on a plurality of said screwdrivers (58) for rotating said screws (56), for lifting said insole (50) supported thereby, in relation to said outsole (46).

7. The insole mapping apparatus (10C) according to claim 1, wherein said processor (29) is further configured to determine said at least one deformation (36A1) of the body of said user (19) according to at least one member selected from a group consisting of: a biofeedback sensor (91), two biofeedback sensors (91) each disposed under one foot of the user, manual inputs according sensation of said user (19), cameras (31) diagnosing walking patterns.

8. A shoe (10A), comprising:
an outsole (46);
a plurality of nuts (54) fixed to said outsole (46);
a screw (56) threaded through each of said nuts (54) and comprising a head (57), being accessible from a bottom of said outsole (46); and
an insole (50) being supported by said screws (56),
thereby allowing disposing said bottom of said outsole (46) on a plurality of screwdrivers (58) for rotating said screws (56), for lifting said insole (50) supported thereby, in relation to said outsole (46).

9. A shoe (10A) according to claim 8, further comprising:
an insole mapping apparatus (10C) for determining an insole map (74), and for operating said screwdrivers (58) according to said insole map (74).
